(19) United States
(12) Patent Application Publication
(10) Pub. No.: US 2010/0029911 A1
(43) Pub. Date: Feb. 4, 2010

(54) Method For Solid-Phase Peptide Synthesis And Purification

(75) Inventors: Hans-Georg Frank, El Kerkrade (NL); Monika Casaretto, Mönchengladback (DE); Karsten Knorr, Krefeld (DE)

Correspondence Address: VENABLE LLP, P.O. BOX 34385, WASHINGTON, DC, 20043-9998, US

(73) Assignee: AplaGen GmbH, Baesweiler (DE)

(21) Appl. No.: 11/791,342

(22) PCT Filed: Nov. 24, 2005
(86) PCT No.: PCT/EP2005/012576

§ 371 Date: May 23, 2007

(30) Foreign Application Priority Data
Nov. 24, 2004 (EP) 04027817.8
Nov. 24, 2004 (EP) 04027840.0
Mar. 16, 2005 (IB) PCT/IB05/00675

(51) Int. Cl. C07K 14/00; C07D 471/04

(52) U.S. Cl. 530/402; 546/88

(57) ABSTRACT

Use of an activated solid phase and a peptide-conjugated anchoring part for solid phase peptide synthesis, wherein the anchoring part is coordinatively and reversibly attached to the activated solid phase.

The present invention relates to methods of solid-phase peptide synthesis and to compounds that can be used for such methods. In particular, it refers to peptidic compounds comprising a metal chelating moiety.

Chemical synthesis of peptides is well established. In principle, two different methods may be distinguished. The synthesis in solution is often very time consuming and therefore not useful for scientific research, whereas the synthesis on a solid support allows a fast optimization of reaction cycles. The protocols available for solid phase peptide synthesis (SPPS) are based on the Merrifield technique (Merrifield, R. B., J. Amer. Chem. Soc. 85, 1963, 2149) for synthesizing peptides with defined sequences on an insoluble solid support. The support offers the benefit of handling separation from soluble reagents more easily by simple filtration. The SPPS has developed extremely fast to a number of variants (generically referred to below as Merrifield-type synthesis or SPPS).

Whilst synthesis of small peptides (up to 30meres) is usually of no problem to SPPS techniques, there are limitations with peptides at a size of 40 up to 120 (or even more) amino acids. Their properties correspond much better to the term “small protein” than to the term “large peptide”:

a) Peptides of such bigger size usually also have a secondary structure (e.g. helix, beta-sheet) and a tertiary structure (e.g. leucine-zipper, disulfide bridging of domains) to be constructed beyond synthesis of the linear amino acid sequence. While the currently known variants of the SPPS technique aim solely at the build up of the primary structure, they do not provide any tool to control the intramolecular formation of secondary and tertiary structures in a suitable way. Upon simultaneous detachment of all products from the resin and removal of protective groups, this often results in functionally useless multimolecular aggregates which do not show any desired biological activity.

b) In case of the synthesis of large peptides, it is commonly preferred to synthesize and couple (protected, partially protected or unprotected) peptide fragments instead of cyclic attachment of single amino acid residues on a solid phase over the full length of the peptide sought to be synthesized. Usually, small fragments are either connected by ligation or fragment condensation coupling techniques in solution. The classical SPPS does not provide a simple approach to bring both reaction partners in solution and even reattach them during the next step of the cycle.

c) It is often difficult to control the desired density of starting residues on a polymeric support and the loading of the resin with the first amino acid often goes along with racemization. Though many preloaded resins are commercially available, these do not cover all loadings and the best value for a given peptide synthesis problem often is hard to find.

Metal affinity had so far only rudimentary application in synthesis of peptides, which is documented in the publication of Comely, A. et al. (Journal of the Chemical Society, 2001, 2526-2531, Transition metal complexes as linkers for solid phase synthesis: chromium carbonyl complexes as linkers for arenes). These authors complexed an amino acid with chromium using aromatic ?-electron-systems e.g. as present in the side chain of derivatized phenylalanine. These complexes were produced in solution and the pre-existing chromium complex was then anchored to a solid phase and one synthesis cycle was performed successfully. While the basic applicability of metal complex anchoring to a solid phase was demonstrated, the procedure differs in the following aspects from the invention presented here.

Comely et al. attach the metal ion to the soluble part of the system first and then anchor the complex to a solid phase in the second step. Comely et al. use complexes formed by aromatic ?-donor systems and requires inert atmosphere to protect some of the reagents used This technique has multiple disadvantages: There is leakage of peptide thus attached, yield not exceeding 90% per amino acid coupling step. Hence maximally decameres can be synthesized in technically acceptable yield. Elution of the complex with competitors finally elutes the peptide complex always with chromium in stoichiometric amounts being attached to the peptide, due to the fact that the chromium is more strongly coordinatively attached to the peptide than to the solid phase. For pharmaceutical use of a peptide, this unacceptable. To circumvent this, Comely employs a harsh cleavage procedure that is slow and destructive to the resin to set the peptide free (48 h oxidation of the grounded polymer with air under white light in DCM).

While no other use was made so far of metal complexes in the field of chemical synthesis of peptides, metal affinity resins per se are known in the area of downstream processing of biotechnologically produced, recombinant biomolecules. Metal affinity chromatography has become popular as a tool for chromatographic purification of naturally occurring proteins from crude biological mixtures or fluids, in parallel to traditional comparable techniques such as ion exchange chromatography. Such purification strategy is described e.g. in EP-A-253303 and U.S. Pat. No. 4,877,830 using agarose-derivatives containing iminodiacetate and nitrilotriacetate. Moreover, peptides containing metal-affinity side chains were engineered with the intention to use the side-chain fixed transition metal complexes as luminescent labelling reagents for a given peptide (e.g. WO-A-9603651A1; EPA-A-0178450).

It is the object of the present invention to establish another, flexible method for solid phase synthesis, purification and refolding/deaggregation of small and large peptides including preferably, but not being limited to, peptides with above 30, 60 or 100 amino acids. This object is achieved by the methods or products according to the independent claims.

According to the method of the present invention for solid-phase peptide synthesis, refolding/deaggregation or purification of a peptide, said peptide having an anchoring part is bound to an activated solid phase comprising a solid support, metal chelating ligands covalently bound to the solid support and metal ions Mⁿ⁺ with n=1 to 3 coordinatively bound to said metal chelating ligands, said activated solid phase providing coordination sites for the coordinative and reversible attachment of an anchoring part of said peptide, characterized in that said peptide is of formula I

P—X-L I

wherein P is the peptidyl part which optionally may comprise further non-peptidic moieties or protection groups, X is a linker or amino acid protection group with the proviso that X is not an amino acid monomer or peptide, and L is a metal chelating group, X and L together constituting the anchoring part. Likewise, such anchoring part may be termed a molecular ‘TAG’ according to the present invention.

For the prevention of misunderstanding, it is noted that formula I is not be construed as to refer to C-terminal linkage to a linear peptide but to any sort of N-terminal (N-), C-terminal (C-) or side chain linkage to such peptide.

The activated solid phase is referred to and is construed in the present context, as a “metal affinity resin”, too.

A further object of the present invention are such peptides of formula I, and their anchoring parts X-L, respectively. The methods and use of the present invention relating to such peptides apply likewise to said anchoring parts.

In short, the present invention relies on metal complexes for attaching a peptide structure, preferably a growing peptide, to an appropriate solid support or resin during SPPS, purification or refolding, preferably during SPPS. Such metal complexes can be used during the repetitive synthesis steps and allow e.g. of detaching the growing peptide chain during the coupling step for a segment condensation interlude and reattaching it for again before the following conventional SPPS steps. Moreover, the same principle of anchoring the peptide chain reversibly to a solid support can be used to control and perform folding of a purified peptide product after synthesis. For this purpose, the product is purified and reattached to a metal affinity resin at a suitably diluted relation of a number of product molecules to resin surface, optionally in the further presence of denaturing agents or influences such as e.g. chaotropic salts, in particular urea, detergents, denaturing pH/temperature and/or suitable solvents. If chosen correctly, this will statistically avoid that product molecules can meet each other and thus lead to a preference for intramolecular instead of intermolecular folding (aggregation). Having been attached and purified, the product is then treated with a number of solvents which allow of a gradual re-folding of the molecule and support the correct intramolecular configuration of the peptide chains. The folding steps can be sealed by the formation of e.g. disulfide bonds in the correct position. At the end of this procedure the stabilized product is released correctly folded into—usually aqueous—surrounding. By implementing this common principle, the invention discloses TAG's or anchoring parts, which have to be understood as organic metal chelating groups combined with suitable linkers, them together making up for the anchoring part of the peptide. Such TAG's can be attached to amino acid side chains, carboxy- or amino-groups of a given peptide and introduce metal chelating properties to the site at which they are attached. Procedures for introduction, use and chemical cleavage of such TAG's are disclosed and assembled into a process of peptide synthesis, which includes the steps of synthesis of the peptide sequence, detachment from a metal-affinity resin by chemical cleavage, purification on a metal affinity resin, refolding on a metal affinity resin and chemical release of a TAG-free peptide at the end of the process.

The metal coordination complex formed by the present invention here is characterized by strong chelation of the metal ion to the solid phase and easily reversible, reversible here meaning weaker, chelation with the peptide chain. Accordingly, and in contrast to Comely et al. (supra), firstly metal ions are attached to a solid phase and secondly the growing peptide chain is anchored to the solid phase. This ends up in elution of the peptide without substantial amounts of metal ions being attached to it.

The basic methodology of non-covalent resin attachment by using metal chelating moieties providing free electrons orbitals from heteroatoms such as e.g. N, O, P for coordinative binding of metal ions has already been described and generically claimed in unpublished application PCT/EP2004/00568 from the same applicants. The activated solid phase is referred to and is construed as a “metal affinity resin”, too.

In a preferred embodiment the peptide is a “growing peptide” and subject to peptide elongation procedures, preferably by FMOC chemistry. This implies that the peptide is protected both in susceptible side chains and N-terminally (and eventually C-terminally, where required). The use of amino acid protection groups, in particular amino acid side-chain protection groups, is well-known in the art and is described e.g. in Bodansky, M., Principles of Peptide Synthesis, 2^{nd} ed., Springer Verlag Berlin/Heidelberg, 1993; further, details of Merrifield-type synthesis, coupling reagents, coupling additives and reaction conditions can be found therein. In the context of the present invention, the terms ‘amino acid’ as always meaning ‘ ’a-amino acid in the present context, peptide ‘backbone’, ‘a-amino’ group and ‘side chain’ in respect to an amino acid or amino acid derivative is used in compliance with the respective IUPAC-IUB definition (International Union of Pure and Applied Chemistry and International Union of Biochemistry/Joint Commission on Biochemical Nomenclature, “Nomenclature and Symbolism for Amino Acids and Peptides”, Pure Appl. Chem, 56, 595-624 (1984)).

Preferably the ‘growing’ peptide consists of at least one amino acid to which further suitably protected amino acids or oligopeptides, preferably protected di- or tripeptides comprising hmb-und pseudoproline dipeptides or dipeptides derivatives, or diglycidyl peptides, are added in cyclic or sequential reaction mode or scheme to elongate said peptide. Such sequential scheme is commonly termed ‘Merrifield-type’ solid phase synthesis. In another preferred embodiment, mono- or oligomeric amino acids are added to the N-terminus (N) of the “growing peptide” in a Merrifield type sequential reaction schedule, preferably in a strict (C? N) Merrifield-type sequential reaction schedule or scheme, preferably a Merrifield reaction schedule based on Fmoc-protection group chemistry for the N-terminus. In a further preferred embodiment the mono- or oligomeric amino acids are/or comprise natural and/or unnatural amino acids such as e.g. D-amino acids or L-Nor-lysine for example. Furthermore, the appropriately protected amino acid derivatives or oligomeric fragments being attached in each cycle of the Merrifield-type sequential reaction schedule can be chosen freely.

Any support known to the skilled person in the art of peptide chemistry may be used for the present invention. Preferably the solid support is based on silica, glass or cellulose or a polymer selected from the group consisting of polystyrene resins crosslinked with divinylbenzene, melamine resins and polyvinyl alcohol-based resins. Said supports of the present invention are covalently derivatized with a suitable metal chelating ligand to constitute the solid phase capable of and being activated by the presence of metal ions according to the present invention.

In a further preferred embodiment the suitably polymeric solid support contains ferromagnetic particles.

In a further preferred embodiment separation of liquid and activated solid phase during synthesis cycles is achieved for example by sieving, size-based separation, centrifugation or magnetic particle separation technology. Reactive functional groups can be introduced to the solid support by means of reaction with pre-existing moieties of the solid support or—in the case of polymers—also by copolymerisation with suitably derivatized copolymers.

In a preferred embodiment each metal chelating ligand on the solid support and the chelating group L on the peptide comprises at least one nitrogen, oxygen, phosphor or sulfur atom which is able to establish a coordinative ligand-metal bond. The metal chelating ligands of the solid phase are, directly or by means of linker groups, covalently bound to the solid support. Suitable linker groups are for example amino, carboxy, methylene, oxy, methylenedioxy, polymethylenedioxy, ethylenedioxy and polyethylenedioxy groups.

Beyond the aspect of providing a suitable weak/strong complexation pairing for complex formation of a peptide with a solid phase according to the present invention, essentially the terms ‘chelating ligand’ and ‘chelating group’ do chemically relate to the same class of chelators.

To the skilled person, it is feasible to identify multiple organic moieties which are able to coordinatively bind metal ions e.g. from databases which can be used to find appropriate metal chelating moieties for the metal chelating ligands according to the teaching and framework of the present invention. Example the respective data is given with exemplary data for groups suitable to perform this invention as described herein selected from publicly accessible database is given in Table 1 below:

TABLE 1
Complex association constants of examplary ligand/metal ion combinations

| | Association | pK | | | | | |
|---|---|---|---|---|---|---|---|
| Ligand | step m | Mn2+ | Fe2+ | Co2+ | Ni2+ | Cu2+ | Zn2+ |
| Imidazole | 1 | 1.25 | 1.81 | 2.44 | 3.01 | 4.21 | 2.55 |
| | 2 | 2.3 | 3.04 | 4.34 | 5.53 | 7.72 | 4.89 |
| | 3 | 3.23 | | 5.76 | 7.5 | 10.57 | 7.17 |
| | 4 | | | 6.7 | 8.8 | 12.6 | 9.18 |
| 1-Methylimidazole | 1 | 1.34 | | 2.29 | 3.05 | 4.22 | 2.38 |
| | 2 | 2.08 | | 4.25 | 5.95 | 7.76 | 4.92 |
| | 3 | 3.08 | | 5.32 | 7.61 | 10.65 | 6.6 |
| | 4 | | | 6.7 | 9.13 | 12.86 | 9.21 |
| Iminodiacetic acid | 1 | 4.72 | 5.8 | 6.97 | 8.3 | 10.56 | 7.15 |
| | 2 | 7.82 | 10.1 | 12.2 | 14.7 | 16.3 | 12.4 |
| | 3 | | | | | | |
| Nitrilotriacetic acid | 1 | 7.27 | 8.9 | 10.38 | 11.51 | 12.7 | 10.45 |
| | 2 | 10.44 | 11.98 | 14.33 | 16.32 | 17.43 | 14.24 |
| Ethylenediaminetetraacetic acid | 1 | 11.1 | 14.3 | 16.5 | 18.4 | 18.8 | 16.5 |
| Ethylenediamine | 1 | 2.6 | 4.13 | 5.5 | 7.3 | 10.49 | 5.69 |
| | 2 | 4.5 | 7.32 | 10.1 | 13.44 | 19.6 | 10.64 |
| | 3 | 5.21 | 9.12 | 13.4 | 17.51 | | 13 |
| 1,3-Diaminopropane | 1 | | | | 6.31 | 9.7 | |
| | 2 | | | | 10.6 | 16.74 | |
| | 3 | | | | 12.9 | | |
| trans-1,2-Diaminocyclohexane | 1 | 2.94 | | 6.37 | 7.9 | 11.09 | 6.37 |
| | 2 | 5.33 | | 11.74 | 14.3 | 20.8 | 11.98 |
| | 3 | | | 15.22 | 20 | | 14.1 |
| 1,4,7-Triazacyclononane | 1 | 5.8 | | 11.2 | 13 | 15.6 | 11.5 |
| | 2 | | | | | 27.7 | |
| 1,4,7,10-Tetraazacyclododecane | 1 | | | 14.1 | 16.4 | 24.6 | 16.2 |
| Pyridin | 1 | 0.24 | 0.7 | 1.22 | 1.88 | 2.54 | 1.05 |
| | 2 | | 0.9 | 1.8 | 3 | 4.38 | 1.45 |
| | 3 | | | | 3.3 | 5.7 | |
| | 4 | | | | | 6.03 | |
| 2,2′-Dipyridyl | 1 | 2.6 | 4.2 | 5.8 | 7.04 | 8.12 | 5.12 |
| | 2 | | 7.9 | 11.3 | 13.86 | 13.63 | 9.63 |
| | 3 | | 17.2 | 16 | 20.16 | 17 | 13.3 |
| 2,2′:6′,2″-Terpyridine | 1 | 4.44 | 7.1 | 9.5 | 10.7 | 12.3 | 6 |
| | 2 | | 20.7 | 18.6 | 21.8 | 19.1 | |
| 2,4,6-Tri(2-pyridyl)-1,3,5-triazin | 1 | | 12.3 | | | | |
| 4-Hydroxypyridine-2,6- | 1 | | 6.7 | | 8.4 | 9.2 | 12.2 |
| dicarboxylic acid | 2 | | | | 16.2 | 17.3 | 22.1 |
| 1,10-Phenanthroline | 1 | 4.09 | 5.85 | 7.1 | 8.7 | 9.13 | 6.38 |
| | 2 | 7.52 | 11.15 | 13.7 | 16.8 | 15.84 | 12.08 |
| | 3 | 10.2 | 21 | 19.68 | 24.4 | 21.03 | 17.1 |
| 2-Methyl-1,10-phenanthroline | 1 | 3 | 4.2 | 5.1 | 5.95 | 7.4 | 4.96 |
| | 2 | 5.5 | 7.9 | 10 | 11.8 | 13.8 | 9.36 |
| | 3 | 7.9 | 10.8 | 13.9 | 16.7 | 16.95 | 12.7 |

pK values at 25° C. The pK value is definded as the negative decadic logarithm of the complex association constant K. In each column the pK for each consecutive association step of the metal ion or its complex MLₘ₋₁ with an additional ligand molecule L (MeLₘ₋₁ + L [symbol] MLₘ, K = c(MLₘ)/(c(MLₘ₋₁) * c(L)) is shown. Taken from A. E. Martell, R. M. Smith, R. J. Motekaitis, “Database 46; NIST Critically Selected Stability Constants of Metal Complexes, Version 7”, Texas A&M University, College Station, TX (2003)

Thus, data on the complex formation of different metal ions with chelating molecules are known and can be found in the primary literature and databases. Chelate complex formation reactions are usually fast, attachment being completed in the presence of either organic or aqueous solvent within a few minutes.

More preferably, the chelating ligand or chelating group comprises 1 to 10 of said N, O, P or S-atoms. Examples are carboxy, amino, phosphoryl, sulfonyl, heterocyclic nitrogen, aza, hydroxyl, mercapto. Preferably, it is a nitrogen comprising group or moiety selected from the group consisting of amino, hydroxyl, carboxyl, mercapto, imidazolyl, N-methylimidazolyl, aminopurinyl moieties, phenanthrolyl moieties, pyridyl moieties, bispyridyl moieties, terpyridyl moieties, triazacyclononanonyl moieties, tetraazacyclododecanyl moieties, iminodiacetic acid moieties, nitrilotriacetic acid moieties and ethylenediaminetetraacetic acid moieties.

Further preferred examples of chelating groups or ligands, supposing an admissible pairing of chelating group and ligand according to the present invention, are triphenylphosphine moieties, aminopurine moieties, preferably 6-aminopurine moieties, phthalocyanine moieties, 1,10-phenanthroline moieties, preferably 5-amino-1,10-phenanthroline moieties, terpyridine moieties, preferably 4′-amino-[2,2′;6′,2″]terpyridine moieties, triazacyclononane moieties, preferably [1,4,7]triazacyclononane moieties and tetraazacyclododecanyl moieties, preferably [1,4,7,10]tetraazacyclododecane moieties.

Preferably, the activated solid phase is comprising a solid support, metal chelating ligands bound to the solid support and metal ions Mⁿ⁺ with n=1 to 3 coordinatively bound to said metal chelating ligands, said activated solid phase providing coordination sites for the coordinative and reversible attachment of an anchoring part of a peptide, wherein said metal chelating ligands are methylene-aminopyridine groups of formula V

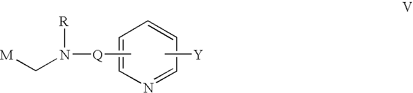

wherein M is the solid support, Y is H or C1-C4 alkyl, Q is either (i) —CH2-, —NH— or —C2H4- or (ii) —(C2H3R′NR′)ₓ—CH2- with the proviso that each R′ is H or CH3 and x is 1 or 2 or (iii) is eliminated such as that the nitrogen is directly bonded to the pyridyl moiety, and further wherein independent of Q, radical R is (a) H, C1-C4 alkyl or C2-C4 hydroxyalkyl, and wherein when Q is —CH2-, radical R can additionally be either (b.) allyl, benzyl or o-hydroxybenzyl or (c.) —C2H3R′NR′)y-CH2-pyridyl-Y with the proviso that each R′ is H or CH3 and y is 0 or 1 or (d.) —CH2)ₘ—OY with the proviso that is m is 2 or 3 or (e.) —C2H3R′NR1R2 with the proviso that R′ is H or CH3, R1 is H, C1-C4 alkyl, C2-C4 hydroxyalkyl, phenyl or benzyl, and R2 is H, C1-C4 alkyl or C2-C4 hydroxyalkyl, or (f.) C3H4SR′″ with the proviso that R′″ is C1-C4 alkyl, or (g.) —CₙH₂ₙCOOY with n=1 or 2, or (h.) —CₙH₂ₙSO₃⁻ with n=1 or 2, or (i.) —CH2Z with the proviso that Z is —CONH2 or —NHCONH2.

An even more strongly preferred example of suitable metal chelating ligands covalently bound to the solid support M are picolylamine groups of formula II

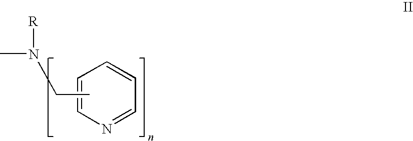

wherein n is 1 or 2, with the proviso that R=H. This meaning that where n=2, formula II would encompass a secondary amine. The aralkyl moiety may be any stereoisomer of methylpyridyl, which is picolyl, such as -2-picolyl, -3-picolyl or -4-picolyl, also routinely coined a-, β- and ?-picolyl, respectively. More preferably the chelating ligand is a 2-picolylamine resin, most preferably it is a bidentate bis-(2-picolylamine) (n=2). Examples of such resins are described in U.S. Pat. No. 4,098,867. In particularly with Cu²⁺ and Ni²⁺ ions, the picolylamines and in particular bis-picolylamine ligands provide very tight binding of the metal ions, allowing of paring such chelating ligand on the solid support with a still comparatively weaker but in absolute terms quite strong chelating group L on the peptide, allowing of reversible but tight attachment of the peptide to the activated solid phase. An example of such resin is Dowex M-4195 (Dow Chemicals, U.S.A.), a macroporous chelating resin consisting of a bis-2-picolylamine functionality attached to a styrene-divinylbenzene polymeric matrix. Dowex M-4195 is capable of removing a number of transition metal cations in the 3d series (Ni,Co,Fe) but shows a particular affinity for copper (Diniz et al., ‘Uptake of heavy metals by chelating resins from acidic manganese chloride solution’, Minerals and Metallurgical Processing 17, 217, 2000).

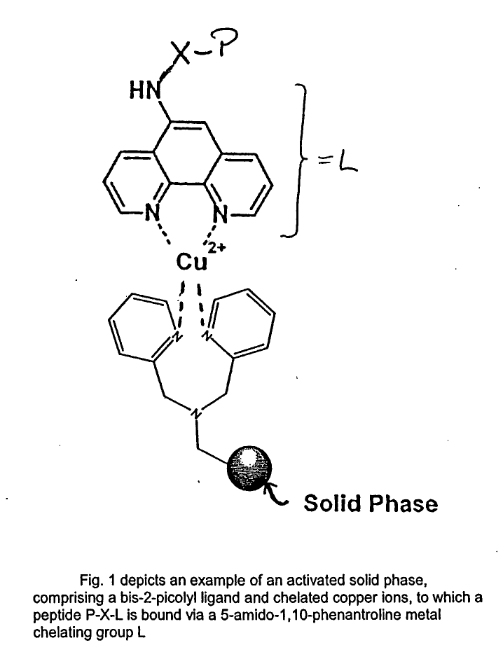

The terms strong/weak refer to complex stability constants, of course. Particularly suitable for pairing with the preferred picolylamine ligands with which the solid phase M is decorated, in particular bis-(picolylamine) ligands and most preferably bis-(2-picolylamine) ligands, are 5-amino-, glycine-5-amino- or 5-amido-1,10-phenantrolines on the peptide side. Such combination is particularly preferred according to the present invention, as is shown exemplarily in FIG. 1. Preferably, the complex metal cation M²⁺ in such combination is Ni²⁺ or Cu²⁺, most preferably it is Cu²⁺.

In a preferred embodiment, an anchoring part of the peptide may comprise 1 to 10 of said nitrogen, heterocylic nitrogen, aza, azido, oxygen, phosphor or sulfur-containing chelating groups L, the latter which are preferably concatemerized, possibly with suitable spacers such as e.g. alkyl or polyethylenglycol chains. It may also be possible, according to the present invention, that the peptide may comprise more than one, preferably two different, anchoring parts.

In another embodiment of the invention the coordinative bond of the metal chelating ligands of the activated solid phase to the metal ions is stronger than the coordinative bond of the anchoring part of the peptide, that is the chelating group L, to said metal ions in terms of complex stability constant.

More preferably the chelating group L is selected from the group consisting of amino, hydroxyl, carboxyl, mercapto, imidazolyl, N-methylimidazolyl, aminopurinyl moieties, phenanthrolyl moieties, pyridyl moieties, bispyridyl moieties, terpyridyl moieties, triazacyclononanonyl moieties, tetraazacyclododecanyl moieties, iminodiacetic acid moieties, nitrilotriacetic acid moieties and ethylenediaminetetraacetic acid moieties.

Preferably the metal Mⁿ⁺ is selected from the group consisting of Mn²⁺, Cu²⁺, Ni²⁺, Co²⁺, Zn²⁺, Mg²⁺, Ca²⁺, Fe²⁺, Fe³⁺ and lanthanide ions, particularly preferred Mⁿ⁺ is Cu²⁺, Ni²⁺, Co²⁺ and Zn²⁺, most preferably it is Cu²⁺ or Ni²⁺

According to the invention a competitive agent can be added to the anchored peptides in order to competitively detach the anchored part of the peptide from the activated solid phase. Preferably the competitive agent is added to the reagent mixture of the coupling step of a Merrifield-type reaction schedule.

A suitable competitive chelating agent or ligand has about the same or weaker affinity for the free coordination sites at the activated solid phase as each individual metal ion chelating moiety of the anchoring part of a peptide to said coordination sites. Detachment is achieved by adding a large excess (typically 10²-10⁶ molar excess of competitive ligand related to the attached ligand) of competitive ligand compared to the attached peptide to the solvent.

In a preferred embodiment the competitive agent is soluble in the reagent mixture of the coupling step and does not react with the ingredients of the reagent mixtures.

In contrast to the detachment, a reattachment of the anchoring part of the peptide to an activated solid phase is possible e.g. by diluting the mixture containing an activated solid phase is, a competitive agent and a non-attached peptide.

In a further preferred embodiment, the competitive ligand contains at least one moiety able to chelate metal ions, preferably a nitrogen containing moiety, selected from the group consisting of imidazolyle, N-methyl-imidazolyle, aminopurine, phenanthroline, bipyridine, terpyridine, triazacyclononane and tetraazacyclododecane, iminodiacetic acid moieties, nitrilotriacetic acid moieties and ethylendiaminetetraacetic acid moieties.

In another preferred embodiment, the competitive ligands contains structural moieties having electron pairs for coordinative bonds such as triphenylphosphine moieties, 6-aminopurine moieties or phthalocyanine moieties.

Specific examples for the competitive ligand are glutathione, ethylenediaminotetraacetic acid, imidazole, N-methyl-imidazole, phenanthrolines, preferably 5-amino-1,10-phenanthrolines, aminoterpyridines, triazacyclononanes or tetraazacyclododecanes. In a preferred embodiment the competitive agent is soluble in the solvent and reagent mixture of the coupling/or washing steps of solid phase synthesis, which typically are dichlorometliane, N-methylpyrrolidone or dimethylformamide, and does not react with the ingredients of the reagent mixtures. It is to be noted that only such solvents provide for solubilization of protected peptides and resins, which usually are not hydrophilic or amphiphilic, as one likes to put it.

Preferably, the peptide comprises at least 25, more preferably at least 30, more preferably at least 60, most preferably at least 100 amino acids.

In another possible embodiment, the peptidyl moiety comprises at least one optionally protected, unnatural amino acids, wherein the characterization as non-natural relates to the non-occurrence of the unprotected amino acid in nature. An example is e.g. D-Phe.

It is of course advantageous to be able to selectively remove/cleave the anchoring part from the peptidyl part of the peptide, either after prior detachment of the peptide from the activated solid support by means of competitive ligand, or right away as a means of detaching the peptide from the activated solid support. For pharmaceutical appliances, the chelating group on the peptide is of course an unwanted immunogen or inhibitor for biological function.

Preferably, the linker is an acid-labile and/or photocleavable linker (described e.g. in U.S. Pat. No. 5,739,386). The linker is non-base labile as regards standard 20% Fmoc 20% piperidine chemistry for deprotection. Such linkers are readily commercially available e.g. from Novabiochem (Merck Biosciences, UK); they are usually used to derivativatize solid supports as to provide resin handles for attachment of a starting amino acids or peptide segment for solid phase synthesis; here they are used to derivatize a peptide or starting amino acid with a chelating group, requiring selective removal from the full length peptide in the aftermath. They may also be used to introduce C-terminal carboxamides to the C-terminus of the peptidyl part upon cleavage (e.g. Fmoc Rink linker, EP-322348). Further examples are:
3-(4-Hydroxymethylphenoxy)propionic acid;
[3-({Ethyl-Fmoc-amino}-methyl)-indol-1-yl]-acetic acid;
4-(2-Bromopropionyl)phenoxyacetic acid;
4-(4-[Bis-(4-chlorophenyl)hydroxymethyl]phenoxy)butyric acid dicyclohexylammonium salt;
4-[4-(2,4-Dimethoxybenzoyl)phenoxy]butyric acid;
4-[4-(Diphenylhydroxymethyl)phenoxy]butyric acid dicyclohexylammonium salt;
4-[4-(1-(Fmocamino)ethyl)-2-methoxy-5-nitrophenoxy)butanoic acid;
p-[(R,S)-a-[1-(9H-Fluoren-9-yl)-methoxyformamido]-2,4-dimethoxybenzyl]-phenoxyacetic acid;
4-Hydroxymethylbenzoic acid;
4-Hydroxymethylphenoxyacetic acid;
4-(4-Hydroxymethyl-3-methoxyphenoxy)-butyric acid;
4-[4-(1-Hydroxyethyl)-2-methoxy-5-nitrophenoxy)butanoic acid;
4-[4-Hydroxymethyl-2-methoxy-5-nitrophenoxy)butanoic acid;
N-Fmoc-N-methoxy-3-aminopropionic acid.
4-[4-(1-Fmoc-amino)ethyl)-2-methoxy-5-nitrophenoxy)butanoic acid.

More preferably, such linker moiety is acid-labile as to require at least 50% trifluoroacetic acid or more for cleavage. Most preferably, such linker is acid-labile even at 3-10% trifluoroacetic acid in an aprotic, polar organic solvent such as N-methyl-pyrrolidone or dichloromethane.

Preferably, alone or in combination with the definitions in the preceding paragraph and with the definitions on the chelating groups and preferred chelating groups for the anchoring part of the peptide, such selectively cleavable linkers are attached C-terminally or via amino acid side chains to the peptide, most preferably via the C-terminus.

Equally preferred is the use of protection groups as are routinely used in peptide synthesis, being inert to standard Merrifield-type peptide synthesis, in particular and favorably to Fmoc synthesis; a broad review of such groups is found in Bodansky, supra. In essence, the present invention converts such protection groups into bi-functional linker-like moieties for transiently attaching a metal chelating group covalently to peptide. Most protection groups may be cleaved off under ‘strongly’ acidic conditions of 50%-80% trifluoroactic acid as applied in global deprotection of peptide and are encompassed by the present definition, as are special protection groups requiring selective chemical cleavage or that are base-labile (e.g. Fmoc; this may be of use when attaching an N-TAG to the finished peptide chain at the end of solid phase synthesis). Preferably, the protection groups are protection groups that are orthogonal to Fmoc chemistry and consequently are non-base labile (meaning they are not susceptible to 20% piperidine in dichloromethane or N-methylpyrrolidone). Notably, in contrast to afore said linkers, a protection group according to the present invention is not labile under ‘mildly’ acidic condition of up to 3-10% trifluoroacetic acid in dichloromethane or N-methylpyrrolidone but is inert to mild acidic cleavage from suitably susceptible resins in conventional covalent linkage (e.g. from 2-chlorotrityl or sieber amide derivatized resins. Such are most of the conventional side-chain protecting groups, e.g. trityl or Boc. Most preferably, such protection group is not labile or removable under ‘strongly’ acidic condition nor under ‘basic’ condition as defined above, hence is orthogonal both to acidic and basic conditions but may be removed by selective chemical cleavage: Examples and particularly preferred embodiments of such are e.g. Allyl (in case of a carboxy function at an amino acid side chain or at the C-terminus) and related Alloc protection groups (e.g. for protecting primary amines), both being selectively removable from peptide by Pd(0)catalyzed transacylation (Gomez-Martinez, N^{a}-Alloc temporary protection in solid-phase peptide synthesis, use of amine-borane complexes as allyl group scavengers, J. Chem. Soc., Perkin Trans. 1, 1999, 2871-2874), the Dde group (Bycroft et al., A novel lysine protecting procedure for SPPS of branched peptides, J. Chem. Soc. Chem. Commun. 1993, p. 778-779), a dimedon derivative that is removable by hydrazinolysis, and functional derivatives thereof (N-1-(4-nitro-1,3-dioxoindan-2-ylidene)-ethyl or Nde group, Kellam et al., Tetrahedron 54, 1998, p. 6817-6832; N-1-(4,4-dimethyl-2,6-dioxocyclohexylidene)-3-methylbutyl, Chan 1995). Functional derivatives include chelating group conjugates which essentially retain or have substantially unaffected the reactivity and selective chemical removability of afore types of protection groups; for the purpose of the present invention, a spacer moiety such as an alkyl, aryl or alkanoyl chain may need to be attached peripherally to such protection groups for bonding the metal chelating group to it. A protection group that is both orthogonal to Boc and Fmoc chemistry, hence is both acid and base resistant when bonded to peptide, favorably preserves this inert character for all of the anchoring part moiety by conjugating to the chelating group in aliphatic, aromatic, ester or amido linkage; such linkage is chemically inert during SPPS. Optionally, and equally preferred is to attach an anchoring part comprising a derivatized protection group N-terminal or as an N-TAG; such N-TAG is attached to or is provided with the last amino acid or peptide segment added during synthesis, and may be bonded to the Na or an amino acid side chain. An example is given in the experimental section with TAG18, 10-(4,4-Dimethyl-2,6-dioxo-cyclohexylidene)-10-hydroxy-decanoic acid [1,10]phenanthrolin-5-ylamide hydroacetate.

Most preferably, the protection group is a protection group of the Dde/Nde-type having a functional moiety of formula III

(—CO)₂C═C(—R)(—OH) III

wherein R is substituted or unsubstituted alkyl and wherein preferably the two carbonyl functions are forming a cyclic structure connected by a —CH2-CR′R″-CH2-, —NR′—CO—NR″— or —CR′═CR″— backbone. Preferably, R′,R″ are then alkyl or, taken together, aryl.

It goes without saying that such protection groups may be combined with the chelating moieties or chelating groups described in the foregoing, except where stated that such chelating moiety is only suited for being used as a chelating ligand on the support due to strength of complex formed with metal ion, as has e.g. been said for the picolylamines.

A further object of the present invention are the isolated compounds X-L, constituting the anchoring part reagent for being subsequently ligated to the peptidic part in order to obtain the peptide of formula I. The definitions in the foregoing apply likewise to this object. Preferably, X is a protection group here, more preferably it is a protection group of the alloc/allyl type or of formula III, most preferably it is of the O-1-(2,6-dioxocyclohexylidene)-ethyl, O-1-(4-nitro-1,3-dioxoindanylidene)-ethyl, O-1-(4-halogeno-1,3-dioxoindanylidene)-ethyl or O-1-(2,4,6-trioxo-1,3-diazinanylidene)-ethyl type which may optionally be further, identically or different, N′ and/or N″-substituted with C1 to C6 alkyl which alkyl may be further substituted or is unsubstituted; such, optionally further N-substituted, 2,4,6-trioxo-1,3-diazinane derivatives of the ‘Dde’-type comprising a functional moiety of formula III have been described in WO 99/15510. Most particularly preferred are Dde-based compounds X-L of formula IV comprising a 5-amido-1,10-phenantroline moiety

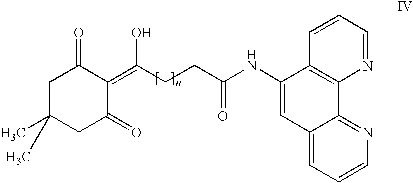

wherein n=1-30, more preferably n=1-20, most preferably n=1-10, which are linkable via the hydroxy group to a primary amino group of a peptide. The activated, vinylogous hydroxy function compares in reactivity to an acid anhydride. Hence Dde, Nde and related compounds are very simple conjugated, not requiring activating coupling reagents.

Furthermore provided is a process for purification of, optionally protected, peptides containing an anchoring part, said anchoring part is coordinatively bound to coordination sites of an activated solid phase comprising a solid support, metal chelating ligands covalently bound to the solid support, metal ions Mⁿ⁺ with n=1 to 3 to said metal chelating ligands, is rinsed, to wash away contaminants such as remnants of protecting groups and scavengers or undesired side products of peptide synthesis.

Another preferred embodiment of the invention makes use of chelating groups attached in final steps of the synthesis to a peptide, namely by having an anchoring part either at the Na of the amino-terminus or proximal to the N-terminus side by attachment to the side chain of at least one of the last 10, preferably the last two, amino acids next to the N-terminus. In this case, purification of the raw product from contaminating side products is achieved by a chromatographic procedure making use of the coordinatively attachment of a peptide to an activated solid phase. This principle can be applied by using regular end-capping of free uncoupled amino groups in each synthesis cycle. In applying this principle, purification of a raw product can be achieved within a single chromatographic run.

Detachment can be achieved by addition of a suitable competition agent as described above or by increasing acidity of the solvent to a critical degree, preferably at least to below pH 6, more preferably to pH 5 or below, especially in aqueous solution, which offers another elegant way of detaching an anchored peptide from a metal affinity resin.

Another advantage associated with the invention is that metal-affinity resins can be reused after peptide synthesis.

Further objects are the uses of an activated solid phase or support comprising methylene-aminopyridine groups of formula V defined above, preferably Bis-picolylamine moieties as defined in formula II above, covalently bound to the solid support as defined above and metal ions Mⁿ⁺ with n=1 to 3 coordinatively bound to said metal chelating ligands, said activated solid phase providing coordination sites for the coordinative and reversible attachment of an anchoring part of a peptide, for solid phase peptide synthesis, peptide refolding/deaggregation or peptide purification. The respective definitions and preferred embodiments as described above apply likewise to such object. In such instance, whilst the use of a peptide of formula I P—X-L is strongly preferred in combination with such this separate object, of course there is no strict need to have a linker or protection group X. Hence anything said in the forgoing on the characteristics of chelating group L carried on the peptide moiety might as well be understood as to relate to peptides of the formula P-L or wherein L is an integral part of the natural structure of P, as for instance is the case with oligohistidine runs in the peptide sequence. The imidazoyl moiety then directly provides for the chelating properties of the peptide moiety P itself, without a need for and hence being devoid of parts X, L. Preferably, in conjunction with the present further object of the invention, the peptide part P comprises, optionally N-terminally protected, at least two imidazolyl side chains in combination with the solid phase or support comprising methylenepyridyl-amine chelating ligands defined above.

In another preferred embodiment said part P comprises oligohistidine or again it is a compound of the type P-L comprising short (1-6 residues) sequences of unnatural amino acids harbouring any of the above mentioned chelating functional groups L may be constituted of, most preferably the unnatural amino acids are having phenanthroline moieties in their side chains with at least one additional amino acid in between, wherein the additional amino acid doesn't interfere or is inert with chelation to the solid support such as for instance, and preferably, glycine. It is also possible, though less preferred, to use any other nitrogen containing group

Preferably said oligohistidine moieties comprises at least 2 histidine residues which are vicinal or spaced apart by not more than 2 amino acid residues; more preferably 6-10 histidine residues. The same applies to imidazolyl side chain comprising amino acids, such as Nor- or Homo-histidine, wherein a Homo-histidine may comprise 1-10 extra methylene groups, to any of which the imidazolyl-moiety may be attached.

In another preferred embodiment said oligohistidine moieties comprise a sequence of at least 2 serial L- or D-histidine residues, more preferably 6 L- or D-histidine residues.

In a further particularly preferred embodiment of the present object of the invention said mono- or oligomeric amino acids of the anchoring part of the peptide contain at least one 5-amino-1,10-phenanthroline moiety or 5-amido-1,10-phenanthroline moiety, and preferably up to 10 therefrom.

In combination with the methylene-aminopyridine resins of the present invention, more preferably with a substituted or unsubstituted 2-picolyl resin, most preferably in combination with a bis-(2-picolylamine) metal affinity resin according to the present invention, this provides of both reversible, non-covalent and still very strong attachment of the peptide via the metal complex to the solid support, whilst at the same time preserving the decisive fact that the solid support must provide the more strongly metal chelating group as compared to the anchoring group on the peptide within such a given pairing. ‘More strongly chelating’ refers to complex stability constant, of course.

It is possible to repetitively detach and reattach the growing peptide chain from and onto the activated solid phase. In a preferred embodiment detachment is achieved during the coupling step of peptide synthesis, while reattachment is induced by dilution of the reaction mixture prior to the rinsing steps preceding the following deprotection. Using these steps, it is possible to use “transient” liquid phase synthesis and its advantages, especially for synthesis of large peptides by fragment condensation approaches.

It is further possible to make use of the activated solid phase of the present invention in peptide purification by synthesizing peptide in a traditional way from C to N-terminus, covalently attached to a solid-phase, but adding a chelating group L and anchoring part L-X, respectively in the last final coupling reaction N-terminally (e.g. a Gly-phenantroline conjugate), only the full length peptide products but not prematurely terminated chains may be easily purified after cleavage from resin by transient attachment to a metal ion activated, chelating solid-phase of the methylene-pyridyl-amine or specifically picolyl-amine type of the present invention.

The invention is further illustrated but not limited to the following examples:

### EXAMPLES

| Abbreviations | Substance |
|---|---|
| DMF | N,N-Dimethylformamide (peptide synthesis grade) |
| DCM | Dichloromethane (peptide synthesis grade) |
| HOBT | 1-Hydroxybenzotriazole (anhydrous) |
| DBU | 1,8-Diazabicyclo[5,4,0]undec-7-en 98% |
| PyBOP | Benzotriazole-1-yl-oxy-tris-pyrrolidino-phosphonium- |
| | hexafluorophosphate |
| DIPEA | N-Ethyldiisopropylamin 98+% |
| TIS | Triisopropylsilan 99% |
| MeOH | Methanol HPLC (gradient grade) |
| TFE | 2,2,2-Trifluorethanol 99.8% |
| EDT | Ethanedithiol |

Note:
The term ‘Dipicolyl-’ is used as a synonym for ‘Bispicolyl-’ below.

### Example 1

### Synthesis of a chemically cleavable Tag for amino groups (Example of an N-TAG: 10-(4,4-Dimethyl-2,6-dioxo-cyclohexylidene)-10-hydroxy-decanoic acid [1,10]phenanthrolin-5-ylamide hydroacetate (TAG18))

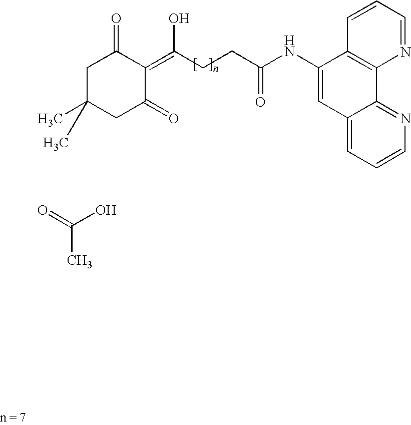

### a) Synthesis of 9-([1,10]Phenanthrolin-5-ylcarbamoyl)-nonanoic acid methyl ester

[1,10]Phenanthrolin-5-ylamine (9.94 mmol, 1.94 g) was dissolved in 250 ml pyridine. 9-Chlorocarbonyl-nonanoic acid methyl ester (12.36 mmol, 2.67 g) was added dropwise and the mixture was stirred overnight. Pyridin was removed by vacuum destillation. The residue was dissolved in DCM, the remaining solid filtered off and the organic phase extracted with saturated NaHCO₃ solution. After drying with Na₂SO₄ the solvent was removed by vacuum destillation. The crude product was purified by flash chromatography (MeOH/CHCl₃ 1:19).

¹H-NMR (400 MHz, DMSO-d₆), d [ppm]:

10.10 (s, 1H), 9.12 (dd, 1H, J=1.5 Hz, J=4.2 Hz), 9.03 (dd, 1H, J=1.6 Hz, J=4.3 Hz), 8.59 (dd, 1H, J=1.4 Hz, J=8.4 Hz), 8.45 (dd, 1H, J=1.6 Hz, J=8.1 Hz), 8.17 (s, 1H), 7.82 (dd, 1H, J=4.2 Hz, J=8.4 Hz), 7.74 (dd, 1H, J=4.3 Hz, J=8.1 Hz), 3.57 (s, 3H), 2.53 (t, 2H, J=7.4 Hz), 2.29 (t, 2H, J=7.4 Hz), 1.69 (q, 2H, J=7.1 Hz), 1.53 (q, 2H, J=6.9 Hz), 1.43-1.25 (m, 8H)

### b) Synthesis of 9-([1,10]Phenanthrolin-5-ylcarbamoyl)-nonanoic acid

9-([1,10]Phenanthrolin-5-ylcarbamoyl)-nonanoic acid methyl ester (25 mmol, 9.84 g) and KOH (25 mmol) was refluxed in 150 ml 1,4-dioxane/H₂O 1:1 for several hours. After cooling NH₄OAc (30 mmol, 2.31 g) is added and the solvent is removed. The residue was washed with 15 mM HCl and dried over P₂O₅. The crude product was purified by flash chromatography (MeOH/CHCl₃ 1:9→1:4).

¹H-NMR (400 MHz, DMSO-d₆), d [ppm]:

10.21 (s, 1H), 9.11 (dd, 1H, J=1.4 Hz, J=4.2 Hz), 9.02 (dd, 1H, J=1.5 Hz, J=4.2 Hz), 8.62 (dd, 1H, J=1.2 Hz, J=8.4 Hz), 8.44 (dd, 1H, J=1.5 Hz, J=8.1 Hz), 8.17 (s, 1H), 7.82 (dd, 1H, J=4.2 Hz, J=8.4 Hz), 7.73 (dd, 1H, J=4.3 Hz, J=8.1 Hz), 2.52 (t, 2H, J=7.7 Hz), 2.13 (t, 2H, J=7.4 Hz), 1.68 (q, 2H, J=7.1 Hz), 1.48 (q, 2H, J=6.8 Hz), 1.43-1.25 (m, 8H)

### c) Synthesis of 10-(4,4-Dimethyl-2,6-dioxo-cyclohexylidene)-10-hydroxy-decanoic acid [1,10]phenanthrolin-5-ylamide hydroacetate

9-([1,10]Phenanthrolin-5-ylcarbamoyl)-nonanoic acid (18.45 g, 7.00 mmol), 4-dimethylaminopyridin (3.68 mmol, 0.45 g), dimedone (20.33 mmol, 2.85 g), and N,N′-dicyclohexylcarbodiimide are dissolved in 100 ml DMF for 2 days. The solvent was removed by destillation and the residue dissolved in DCM. The N,N′-dicyclohexylurea is removed by filtration, the solvent removed in vacuo and the crude product purified by flash chromatography (MeOH/DCM/HOAc 1:19:+1%).

¹H-NMR (400 MHz, DMSO-d₆), d [ppm]:

18.24 (br s, 1H), 9.08 (m, 2H), 8.31 (m, 2H), 8.13 (m, 2H), 7.57 (m, 2H), 3.00 (t, J=7.4 Hz, 2H), 2.55 (7.5 Hz, 2H), 2.51 (s, 2H), 2.31 (s, 2H), 1.81 (m, 2H), 1.61 (m, 2H), 1.30-1.45 (m, 8H), 1.03 (s, 6H)

HR-ESI-MS:

| | |
|---|---|
| Calculated: (C₃₀H₃₄N₃O₄): | 500.25548 |
| Found: | 500.25523 |

Examples 2-5 Describe the Syntheses of Organic Compounds which Harbour Suitable—Partially Protected—Chelating Groups and which can be Coupled in Analogy to Example 1c to Form Dimedone-Based N-Terminal TAG's:

### Example 2

### Synthesis of (Bis-tert-butoxycarbonylmethyl-amino)-acetic acid [TAG8b]

### a) Synthesis of (Bis-tert-butoxycarbonylmethyl-amino)acetic acid benzyl ester [TAG8a]

20 mmol Glycine benzyl ester p-tosylate, 40 mmol Bromo-acetic acid tert-butyl ester and 60 mmol DIEA are dissolved in 35 ml dry DMF. The reaction mixture is vortexed for 4 days. Precipitated salts are filtered off and the solution is dissolved in 250 ethyl acetate. The organic layer is extracted with the following solutions: 2×200 ml 1N NaOH, 3×1N NaOH/brine 1:1. The solution is dried with Na₂SO₄, the solvent removed by vacuum destillation and the crude product is purified by flash column chromatography (ethyl acetate/hexane 1:4).

¹H-NMR (400 MHz, DMSO-d₆) ppm 7.42-7.29 (m, 5H), 5.10 (s, 2H), 3.60 (s, 2H), 3.50 (s, 4H), 1.38 (s, 18H); LC-MS (ESI): (M+H)⁺=394

### b) Synthesis of (Bis-tert-butoxycarbonylmethyl-amino)-acetic acid [TAG8b]

17.5 mmol (Bis-tert-butoxycarbonylmethyl-amino)-acetic acid benzyl ester is dissolved in 75 ml THF and the catalyst palladium, 10% on charcoal, 0.70 g, is added. The reaction vessel is flushed several times with hydrogen and the reaction mixture is stirred under hydrogen until the hydrogen consumption stops. The catalyst is filtered off over celite and the solvent is removed in vacuo.

¹H-NMR (400 MHz, DMSO-d₆) ppm, 12.2 (br s, 1H), 3.46 (s, 2H), 3.44 (s, 4H), 1.40 (s, 18H); LC-MS (ESI): (M+H)⁺=304

### Example 4

### Synthesis of Fmoc-Gly-5-Amino-1,10-phenanthroline (TAG5)

2 ml DIEA, 6 mmol Cl-HOBt and 12 mmol DIC are added to a solution of 2.56 mmol Fmoc-Gly-OH in a minimum of DMF. This solution is vortexed for 5 minutes and a solution of 0.5 g 5-Amino-1,10-phenantroline in a minimum of DMF is added. The mixture is incubated for 12 hours and diluted with the 5fold volume of ethylacetate. The solution is washed three times with sodium hydrogencarbonate. The crude product precipitates, is filtered off and purified by column chromatography or LCMS. The crude peptide was dissolved in DMSO and 1000 μl purified on a Gilson Nebula LCMS System using a Kromasil RP C18 column. The linear gradient extended from 5% aqueous TFA (0.1%) to 50% acetonitrile (containing 0.085% TFA) over 50 min. The flow rate was 20 mL/min and the absorbance monitored at 214 nm.

### Example 5

### Synthesis of 4-([1,10]Phenanthrolin-5-ylcarbamoyl)-butyric acid [TAG12b]

### a) Synthesis of 4-([1,10]Phenanthrolin-5-ylcarbamoyl)-butyric acid methyl ester [TAG12a]

In an argon atmosphere 5 mmol 5-amino-[1,10]-phenanthroline are dissolved in 25 ml dry DMF. 6.5 mmol DIEA are added and 5.5 mmol 4-chlorocarbonyl-butyric acid methyl ester are added drop by drop. After stirring for 1 h the solvent and excess base are removed by vacuum destillation. The crude product is purified by reversed phase column chromatography (Merck Lobar RP18 column, eluant: acetonitrile/ammonium hydrogencarbonate, 5 weight-%, gradient: 20% to 40%)

¹H-NMR (400 MHz, DMSO-d₆) ppm 10.11 (s, 1H), 9.13 (dd, J=1.6, 4.3 Hz, 1H), 9.03 (dd, J=1.7, 4.3 Hz, 1H), 8.60 (dd, J=1.6, 8.4 Hz, 1H), 8.44 (dd, J=1.7, 8.2 Hz, 1H), 8.17 (s, 1H), 7.82 (dd, J=4.2, 8.4 Hz, 1H), 7.74 (dd, J=4.3, 8.1 Hz, 1H), 3.63 (s, 3H), 2.59 (t, J=7.2 Hz, 2H), 2.46 (t, J=7.4 Hz, 2H), 1.95 (p, J=7.3 Hz, 2H); LC-MS (ESI): (M+H)⁺=324

### b) Synthesis of 4-([1,10]Phenanthrolin-5-ylcarbamoyl)-butyric acid [TAG12b]

1.5 mmol 4-([1,10]Phenanthrolin-5-ylcarbamoyl)-butyric acid methyl ester are dissolved in 15 ml 1,4-dioxane and 15 ml destilled water. 1.5 ml of 1 N solution of potassium hydroxide are added and the solution is refluxed for 1 hour. The solvent is removed by vacuum destillation and the crude product is purified by reversed phase column chromatography (Merck Lobar RP18 column, eluant: acetonitrile/trifluoroacetic acid, 1 weight-%, gradient: 10% to 20%)

¹H-NMR (400 MHz, CD₃OD) as K salt: ppm 9.11 (dd, J=1.6, 4.3 Hz, 1H), 9.04 (dd, J=1.6, 4.4 Hz, 1H), 8.65 (dd, J=1.5, 8.4 Hz, 1H), 8.41 (dd, J=1.6, 8.1 Hz, 1H), 8.14 (s, 1H), 7.82 (dd, J=4.4, 8.4 Hz, 1H), 7.74 (dd, J=4.4, 8.1 Hz, 1H), 2.64 (t, J=7.6 Hz, 2H), 2.36 (t, J=7.2 Hz, 2H), 2.10 (q, J=7.5 Hz, 2H)); LC-MS (ESI): (M+H)⁺=310

Furthermore tags comprising dicarboxylic acids derivatives other than glutaric acid can be prepared analogously to the here described protocols.

### Example 6

### Coupling of 10-(4,4-Dimethyl-2,6-dioxo-cyclohexylidene)-10-hydroxy-decanoic acid [1,10]phenanthrolin-5-yl-amide [Tag18] to the N-term of a peptide: Tag18-STKKTQLQLEHLLLDLQMILNGINN-CO-NH2

The peptide was prepared following standard procedures on a Rink-amide resin (0.2 mmol). After FMOC deprotection of the last amino acid with piperidine/DMF 1:4 the resin was washed 6 times with 20 ml DMF each. A solution of Tag18*HOAc (0.56 g, 1 mmol) in 20 ml DMF and 1 drop TFA were added to resin and the mixture was shaken for 2 days. The solution was filtered off, the resin washed 6 times with DMF, then 2 times with DCM, and dried in vacuo. Cleavage of the crude peptides is achieved by treatment with 5 ml TFA/TIS/EDT/H₂O (94/1/2.5/2.5) for 120 minutes under inert atmosphere. This solution is filtered into 40 ml cold ether. The precipitate is dissolved in acetonitrile/water (1/1) and lyophilized.

The success of coupling of the TAG18 to the peptide was confirmed by a Nebula-LCMS-system (Gilson).

ESI-MS:

| | |
|---|---|
| Calculated: ([M + 3H]³⁺): | 1120.6 |
| Found: | 1120.7 |

### Example 7

### Metal Affinity Purification of Tag18-STKKTQLQLEHLLLDLQMILNGINN—CO—NH2 and Subsequent Chemical Cleavage of the Tag from the Peptide :H-STKKTQLQLEHLLLDLQMILNGINN-CO-NH2

The crude product from example 3 was purified on AktaFPLC system:

30 mg of the crude peptide were dissolved in 9 ml ACN/iPrOH/H₂O 5:4:1+1% DIEA+5 mM NMI and injected on a column filled with about 5.6 ml Ni-loaded bispicolyl-aminopropyl silica resin. The column was washed with 70 ml of ACN/iPrOH/H₂O 5:4:1+1% DIEA+5 mM NMI, 60 ml ACN/H₂O 1:1, and the product was eluted with 60 ml ACN/H₂O 1:1+1% 1,3-diaminopropane.

Product containing fractions were united, 2.5% hydrazine-hydrate added, the solution shaken for 30 minutes at room temperature and lyophilized. Analysis on a Nebula-RP-LCMS-system (Gilson) showed exclusively the mass signals for the free peptide H-STKKTQLQLEHLLLDLQMILNGINN—CO—NH₂. Neither UV nor mass signals of the educt could be found.

| | |
|---|---|
| Calculated: ([M + 3H]³⁺): | 959.5 |
| Found: | 959.5. |

### Example 8

### Dipicolyl-aminopropyl-CLEAR-Base Resin

1 g CLEAR-Base (HCl) resin (Peptides International) 0.77 mmol/g was swollen in 20 ml DMF and 132 μl DIEA for 20 min. The resin was filtered by suction and a solution of 589 mg (3.85 mmol) 3-bromopropionic acid activated with 1.964 g (3.77 mmol) PyBop, 520 mg (3.85 mmol) HOBt and 1.3 ml (7.7 mmol) DIEA in 10 ml DMF was added. After 2 h stirring the resin was filtered and washed by DMF, dichloromethane and methanol. The resin was dried in vacuum.

To 1 g of the previously prepared (bromopropyl-aminomethyl)-Clear resin in 30 ml of toluene, 164.4 μl dipicolyl-amine (0.914 μmol) were added and the reaction mixture was refluxed for 5 h. The resin was filtered, washed with dichloromethane, methanol, water, methanol and dried.

### Example 9

### [(Di-2-picolylamino)-propyl]-aminomethyl-polystyrene

5 equivalents of 3-bromopropionic acid (1,492 g) dissolved in 10 ml DMF were coupled to 1 g (1.95 mmol) of (aminomethyl)polystyrene resin 150-300 μm using 4.971 g PyBop, 1.316 g HOBt and 3.3 ml (19.5 mmol) DIEA. After 12 h the resin was filtered off and washed with DMF, dichloromethane and methanol. The resin was dried in vacuum.

To 1 g of (bromopropyl-aminomethyl)polystyrene resin in 30 ml of toluene, 164.4 μl di-(picolyl)amine (0.9141 mmol) were added and the reaction mixture was refluxed for 5 h. The resin was filtered, washed with dichloromethane, methanol, water, methanol and dried.

### Example 10

### Di-2-picolyl-aminopropyl-silica

### (As Used in Example 7)

1 g of 3-chloropropyl-silica (3.2% Cl=0.914 mmol/g 230-400 mesh) was suspended in 30 ml of toluene, 164.4 μl di-(picolyl)amine (0.914 μmol) were added and the reaction mixture was refluxed for 5 h. The resin was filtered, washed with dichloromethane, methanol, water, methanol and dried.

### Loading of Nickel Ions:

A slurry of the resin in 0.1 m sodium citrate buffer pH 2.5 was filled into small column (1.7 ml) equilibrated and treated with a 0.1 m solution of NiCl₂ in the same buffer until saturation was reached. The excess of nickel was removed by washing with buffer, and solutions of 10 mM N-methylimidazole and 10 mM EDTA in this buffer.

### Binding of (Bis-tert-butoxycarbonylmethyl-amino)-acetic Acid, TAG8

The column was equilibrated with acetonitrile:50 mM MES pH 6.4 1:1.2 mg of TAG 8 were dissolved in this solution and injected. TAG8 bound to the resin.

Binding of TAG 8 decorated peptide: The above binding experiment is repeated under the same conditions whilst now using complete, deprotected peptide comprising deprotected TAG8 after global deprotection, that is the compound loaded and bound to the resin now is N,N′-diacetyl-2-amino-acetic acid [peptidyl-Na-10-(4,4-dimethyl-2,6-dioxo-cyclohexylidene)]-decanyl ester. The TAG8-peptide binds to the resin.

### Example 11

### Synthesis and Purification of

Tag 18-GGTYSCHFGKLTWVCKKQGG-NH₂

### Synthesis

For the synthesis an Odyssey Microwave Peptide Synthesizer (CEM) has been used. Single Fmoc protected amino acids were added, with commonly used protection groups (trityl, tert.bu, boc, . . . ) where needed (S, T, Y, C, H, K).

### Resin:

Rink amide resin (polymer laboratories) 0.4 mmol/g, 0.625 g resin (0.25 mmol)

### Reagents:

4 equivalents amino acid derivatives (5 ml of a 0.2 mmol/ml solution in DMF)
4 equivalents PyBOP in DMF, 6 equivalents HOBt in 3 ml DMF
4 equivalents DIEA in 2 ml NMP

### FMOC Deprotection:

10 ml 20% piperidine in DMF

After the last FMOC deprotection step the resin (with Peptide still bound) was transferred into a 100 ml round bottom flask, washed several times with DMF and sucked “dry”.

Coupling of Tag18c*HOAc to N Term of Peptide (PeptideT18)

0.72 g of Tag18c*HOAc (1.28 mmol, 5 equivalents) were dissolved in 50 ml DMF and 240 μl DIEA (1.45 mmol, 1.1 equivalents) were added. The “pH value” of this solution was determined to be about 7 by pH indicator paper “Tritest” (Carl Roth). This solution was added to the resin (DMF wet) and shaken at room temperature for 48 h.

### Cleavage of the Peptide Off the Resin and Deprotection

The resin has been washed 5 times with DMF, then 5 times with DCM. 10 ml of a cleavage cocktail (94% TFA, 1.0% Triisopropylsilan (TIS), 2.5% water, 2.5% 3,6-dioxa-1,8-octandithiol (DODT)) were added and shaken for 2 hours. 10 minutes before stopping shaking, additional 100 μl TIS were added.

The mixture was filtered and the peptide precipitated by dropping the filtrate into 40 ml of cold tert-butyl methyl ether (TBME). The mixture was centrifuged and the precipitated peptide dissolved in ACN/H₂O and lyophilised.

Yield crude product: 440 mg

The UV absorption peak of the tagged peptide PeptideT18 exceeds all other signals. Three of the other peaks can be assigned by the signals in the mass spectrometer (see below).

| Time | masses (detected ions) | explanation |
|---|---|---|
| 26.19 | 2155.4 (719.5/1078.7) | Peptide |
| 32.23 | 2638.5 (660.6/880.5/1320.4) | PeptideT18 |
| 33.77 | 2694.9 (899.3) | +56: tBu adduct |
| 35.35 | 2694.9 (899.3) | +56: tBu adduct |

The relation of the intensities of the MS signals is about 1 to 9. This indicates that the coupling of Tag18 to the peptide had a yield of about 90%.

CFPS Purification CFPS purification was performed by binding the crude peptide to a NiNTA resin and washing off non-tagged compounds in a column followed by cleavage of the tag in a batch hydrazinolysis experiment.

### Solvents/Buffers:

### ACN/H₂O 1:1

One part distilled water was mixed with one part ACN, and degassed by 10 min sonification and bubbling argon through the solvent for 10 minutes.

### ACN/MES 1:1

50 mmol 2-(N-morpholino)ethanesulfonic acid monohydrate (MES) have been dissolved in distilled water and 33 ml of a 1N NaOH solution have been added. The solution was diluted to a total volume of 1 l by adding water (pH=6.8, glass electrode). One part of the buffer was mixed with one part ACN, and degassed by 10 min sonification and by bubbling argon through the solvent for 10 minutes.

### Column

A 5.6 ml Omnifit column (Supelco) was packed with Ni-NTA His-Bind Resin (Merck Biosciences) and equilibrated with ACN/MES 1:1.

### CFPS Purification

41.35 mg of the crude peptide were dissolved in about 7 ml ACN/MES 1:1. This slightly turbid solution was injected manually on the column (not displayed in chromatogram, see below).

At a flow of 5 ml/min the column was washed with ACN/MES 1:1, 30 ml, a gradient of ACN/MES 1:1 to ACN/H₂O 1:1, 30 ml, and with ACN/H₂O 1:1, until the conductivity drops to 0 mS/cm (11.6 ml).

(manual injection of 7 ml sample is not displayed))

A rather small injection peak shows the removal of non-tagged compounds by washing.

### Cleavage of Tag18 by Hydrazinolysis

The resin was transferred into a 50 ml Falcon tube, 6 ml of ACN/H₂O 1:1 and 600 μl hydrazine-hydrate added and shaken for 1 hour. 6 ml ACN/H₂O 1:1 were added and the resin filtered off and washed with 12 ml H₂O. The filtrate was lyophilised, redissolved in ACN and water and lyophilised a second time.

Yield: 18.2 mg

### Removing of Low Molecular Weight Compounds by Diafiltration

Low molecular weight compounds were removed by diafiltration using a stirred cell model 8400 (Amicon/Millipore) with a cellulose acetate membrane with a molecular weight cut-off of 500 g/mol (Amicon YC05 ultrafiltration discs, cellulose acetate, 500 NMWL, 76 mm, Millipore).

14.4 mg of the CFPS purified peptide were dissolved in 1 ml ACN and 1 ml ACN/H₂O/TFA 1:9:0.1%. This solution was diluted with ACN/H₂O 5:95 up to a total volume of 50 ml. This solution was transferred into the stirred cell that has been equipped with the membrane.

The solution was diafiltrated for 6 hours with 200 ml ACN/H₂O 5:95 (i.e. 4 volumes) under a pressure of 3 bar (Argon). The retendate was lyophilized two times.

Yield: 8.9 mg

| Time | masses (detected ions) | explanation |
|---|---|---|
| 30.52 | 2187.4 (730.0/1094.7) | +28 g/mol |
| 31.20 | 2155.4 (719.5/1078.7) | BB57 |
| 31.72 | 2168.4 (723.7/1085.2) | +14 g/mol |
| 32.16 | 2153.2 (718.8/1077.6) | −2 g/mol: disulfide |
| 33.26 | 2210.2 (737.7/1106.1) | +56 g/mol: tBu adduct |
| 34.94 | 2211.4 (738.1/1106.7) | +56 g/mol: tBu adduct |
| 37.03 | 841 | not identified |

(retention times differ form those of the above LC-MS by +5.00 minutes due to technical reasons)

No TAG18-bearing peptide is detectable (neither UV nor MS). All UV peaks show a corresponding MS signal. Besides uncertainties at 37.03 min. no other Tag degradation products are detectable by UV or MS.

1. A peptide having an anchoring part for binding to an activated solid phase comprising a solid support, a metal chelating ligand covalently bound to the solid support and metal ions Mⁿ⁺ with n=1 to 3 coordinatively bound to said metal chelating ligands, for solid-phase peptide synthesis of said peptide, said activated solid phase providing coordination sites for the coordination and reversible attachment of the anchoring part of the peptide, wherein the peptide is of formula I
P—X-L I
wherein P is the peptidyl part which optionally may comprise further non-peptide moieties or protection groups, X is a selectively cleavable linker or orthogonal amino acid protection group wherein X is not an amino acid monomer or peptide, and L is a metal chelating group.

2. The peptide of claim 1, wherein X is a linker or amino acid protection group which is base resistant and is acid-labile and/or photo-cleavable.

3. The peptide of claim 1, wherein X is an orthogonal amino acid protection group which is non-base labile and non-acid labile and which is of the Dde/Nde-type having a functional moiety of formula III
(—CO)₂C═C(—R)(—OH) III
wherein R is substituted or unsubstituted alkyl and wherein the two carbonyl groups form a cyclic structure connected by a —CH2-CR′R″—CH2-, —NR′—CO—NR″— or —CR′═CR″— backbone wherein R′,R″ are alkyl or aryl.

4. The peptide of claim 1, wherein L comprises a phenanthrolyl moiety selected from the group consisting of 5-amino- and 5-amid-[1,10]-phenantroline.

5. The peptide of claim 1, wherein the peptide is a growing peptide chain subject to peptide elongation procedures.

6. The peptide of claim 1, wherein the metal Mⁿ⁺ is selected from the group consisting of Mn²⁺, Cu²⁺, Ni²⁺, Co²⁺, Zn²⁺, Mg²⁺, Ca²⁺, Fe²⁺, Fe³⁺ and lanthanide ions.

7. A peptide of formula P—X-L, wherein P is the peptidyl part which further comprises non-peptide moieties or protection groups, X is an orthogonal amino acid protection group that is resistant to Fmoc- or to Boc-chemistry, X is not an amino acid monomer or peptide, and L is a metal chelating group.

8. The peptide of claim 7, wherein L comprises a phenanthrolyl moiety selected from the group consisting of 5-amino- and 5-amido-[1,10]-phenantroline.

9. The peptide of claim 7, wherein X is an orthogonal protection group which is both base and acid resistant and is a Dde/Nde-type protection group having a functional moiety of formula III
(—CO)₂C═C(—R)(—OH) III
wherein R is substituted or unsubstituted alkyl and wherein the two carbonyl groups form a cyclic structure connected by a —CH2-CR′R″-CH2-, —NR′—CO—NR″— or —CR′═CR″— backbone wherein R′,R″ are alkyl or, taken together, aryl.

10. A compound X-L of formula IV 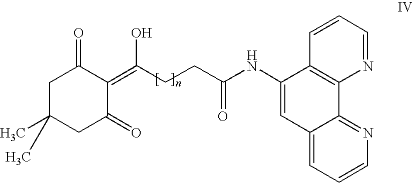
wherein n=1-30.

11. The peptide of claim 1, wherein X-L is a compound of formula IV 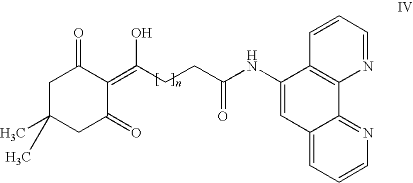
wherein n=1-30.

12. The peptide of claim 1, wherein the metal chelating ligands of the activated solid phase are methylene-aminopyridine groups of formula V 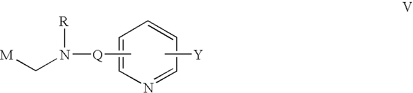
wherein M is the solid support, Y is H or C1-C4 alkyl, Q is either (i) —CH₂—, —NH— or —C₂H₄— or (ii) —(C₂H₃R′NR′)ₓ—CH₂— wherein each R′ is H or CH₃ and x is 1 or 2 or (iii) is eliminated such as that the nitrogen is directly bonded to the pyridyl moiety, and further wherein independent of Q, radical R is (a) H, C1-C4 alkyl or C₂-C₄ hydroxyalkyl, and wherein when Q is —CH₂, radical R can be either (b.) allyl, benzyl or o-hydroxybenzyl or (c.) —(C₂H₃R′NR′)y-CH₂-pyridyl-Y wherein each R′ is H or CH₃ and y is 0 or 1 or (d.) —(CH₂)ₘ—OY wherein m is 2 or 3 or (e.) —C₂H₃R′NR1R2 wherein R′ is H or CH3, R1 is H, C1-C4 alkyl, C2-C4 hydroxyalkyl, phenyl or benzyl, and R2 is H, C1-C4 alkyl or C2-C4 hydroxyalkyl, or (f.) C₃H₄SR′″ wherein R′″ is C1-C4 alkyl, or (g.) —CₙH₂ₙCOOY with n=1 or 2, or (h.) —CₙH₂ₙSO₃⁻ with n=1 or 2, or (i.) —CH2Z and wherein Z is —CONH₂ or —NHCONH2.

13. The peptide of claim 12, wherein a picolylamine group of formula II is bound to the solid support M 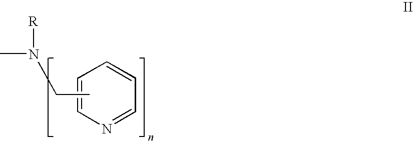
wherein n=1 or 2 and R=H

14. The peptide of claim 6, wherein the metal Mⁿ⁺ is selected from the group consisting of Cu²⁺, Ni²⁺, and Co²⁺.

15. The peptide of claim 5, wherein the peptide is a growing peptide, bound via an anchoring part to a metal ion, which is bound to a metal chelating ligand bound to a solid support and subject to peptide elongation procedures.

16. The peptide of claim 5, wherein mono- or oligomeric amino acids are added at the C- or N-terminus to the growing peptide in a Merrifield-type sequential reaction scheme.

17. The peptide of claim 1, wherein the metal chelating ligand of the activated solid phase is selected from the group consisting of amino, hydroxyl, carboxyl, mercapto, imidazolyl, N-methylimidazolyl, aminopurinyl moieties, phenanthrolyl moieties, pyridyl moieties, bipyridyl moieties, terpyridinyl moieties, triazacyclononanonyl moieties, tetraazacyclododecanyl moieties, iminodiacetic acid moieties, nitrilotriacetic acid moieties and ethylenediaminetetraacetic acid moieties.

18. The peptide of claim 1, wherein the anchoring part of the peptide chain is located at the C-terminus and/or in at least one amino acid side chain of the peptide.

19. The peptide of claim 1, wherein after detachment of the peptide from the activated solid phase by diluting the reaction mixture with a competitive ligand, the peptide is reattached to the activated solid phase.

20. The peptide of claim 1, wherein the non-covalent and coordinative attachment of the metal ion to the metal-chelating ligand of the solid support is stronger than the attachment to the anchoring group on the peptide.

21. A method for solid phase synthesis of peptides by non-covalent attachment of a growing peptide chain to an activated solid phase, wherein the active component of the solid phase is formed by metal chelate complexes having free coordination sites for non-covalent attachment of a growing peptide chain to the activated solid phase via an anchoring part of the growing peptide chain.
